(19) United States
(12) Patent Application Publication
(10) Pub. No.: US 2008/0090916 A1
(43) Pub. Date: Apr. 17, 2008

(54) Compounds for Treating Urinary Incontinence

(76) Inventors: Jeffrey Martin Thompson, Bedfordshire (GB); Hans Jurgen Gruss, Oxon (GB); Alexander Ungar, Cumberland (GB)

Correspondence Address: Leon R. Yankwich;Yankwich & Associates, 201 Broadway, Cambridge, MA, 02141, US

(21) Appl. No.: 11/795,453

(22) PCT Filed: Jan. 25, 2006
(86) PCT No.: PCT/GB06/00256

§ 371 Date: Jul. 17, 2007

(30) Foreign Application Priority Data
Jan. 25, 2005 (GB) 0501580.5

(51) Int. Cl. A61K 31/137; A61P 13/00; C07C 215/04

(52) U.S. Cl. 514/653; 562/448; 564/363

(57) ABSTRACT

The invention relates to compounds of formula (I) or a pharmaceutically acceptable solvate or salt thereof, including a solvate of such a salt, wherein R¹ is a group such that a compound of formula (I) is a prodrug of 1R,2S-methoxamine that is converted within the kidney tubules into its active form or an active metabolite thereof. The compounds find particular use in the treatment or prophylaxis of urinary incontinence.

### INTRODUCTION

The present invention relates to prodrugs of the compound 1R,2S-methoxamine (formula (Ib)). It also relates to pharmaceutical compositions containing such prodrugs.

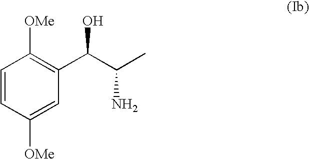

### BACKGROUND OF THE INVENTION

Urinary incontinence may be caused by a number of disorders. It is particularly prevalent amongst the elderly, and in women who have suffered injury from pregnancy and childbirth. More than twice as many women as men are affected by the condition. Urinary incontinence may also be caused by genetic conditions.

The various forms of urinary incontinence include urge incontinence, reflex incontinence, overflow incontinence, neurogenic incontinence, post-prostatectomy incontinence, transient incontinence (a temporary condition due to infection or medication) and stress or load incontinence. Some patients may have two or more of these types of incontinence, which is referred to as mixed incontinence. Urge incontinence, which is caused by an overactive bladder, may be caused by the nerve pathways being hyperactive. Stress incontinence is the most common form of urinary incontinence and may, for example, be caused by prolapse of the bladder to a position which puts excessive pressure on the urethral sphincter.

Many of the drugs presently used to combat incontinence have side effect problems which may result in non-compliance with treatment. For some types of urinary incontinence, surgery may be used, but surgery always carries certain risks so may not be advisable for certain patients.

### SUMMARY OF THE INVENTION

The present invention provides a compound of formula (I) or a pharmaceutically acceptable solvate or salt thereof, including a solvate of such a salt, 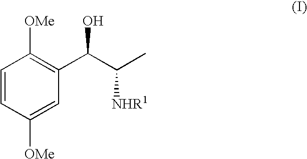
wherein R¹ is a group such that the compound of formula (I) is a prodrug of 1R,2S-methoxamine that is converted within the kidney tubules into its active form or an active metabolite thereof. The compound of formula (I) may be used as a medicament.

In particular, the present invention also provides a compound of formula (Ia) or a pharmaceutically acceptable solvate or salt thereof, including a solvate of such a salt,

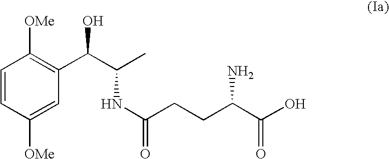

The compound of formula (Ia) may be used as a medicament. The compound of formula (Ia) is a prodrug for 1R,2S-methoxamine. The compound accordingly has use in the treatment or prophylaxis of urinary incontinence.

### BRIEF DESCRIPTION OF THE FIGURES

FIG. 1 shows representative traces of isolated pig vesicourethral junction sphincter muscle strip responses to the compound of formula (III), 1R,2S-methoxamine, and the compound of formula (Ia).

FIG. 2 shows graphs of contractile response of vesicourethral junction sphincter to increasing doses of either 1R,2S-methoxamine, and the compound of formula (Ia) or the compound of formula (III).

FIG. 3 shows representative traces of pig vesicourethral junction sphincter muscle response to various enzyme reaction products.

FIG. 3a shows the contractile response of pig vesicourethral junction sphincter muscle following administration of 50 mM Tris HCl buffer.

FIG. 3b shows the contractile response of pig vesicourethral junction sphincter muscle following administration of 50 mM Tris HCl buffer and gamma glutamyl transferase.

FIG. 3c shows the contractile response of pig vesicourethral junction sphincter muscle following administration of 50 mM Tris HCl buffer, MgCl₂.6H₂O, glycylglycine and the compound of formula (Ia).

FIG. 4a shows the contractile response of pig proximal sphincter muscle following administration of 50 mM Tris HCl buffer, MgCl₂.6H₂O, glycylglycine, gamma glutamyl transferase and the compound of formula (Ia).

FIG. 4b shows the contractile response of pig proximal sphincter muscle following administration of the compound of formula (Ib) at 0.3 mM.

FIG. 5 is a comparative graph of the contractile response of pig bladder neck muscle under the various reaction conditions described for FIGS. 3a-c and 4a-b.

FIG. 6 is a comparative graph of the reaction mixture's effect on vesicourethral junction sphincter muscle strips at 10, 30 and 60 min after incubation.

FIG. 7 shows the effect of the compound of formula (Ia) on vesicourethral junction sphincter muscle strips after a 2-fold dilution post 30 min incubation with the enzyme.

FIG. 8 is a graph showing the effect on vesicourethral junction sphincter muscle strips of the compound of formula (Ib) (0.3 mM) prepared in tris buffer compared to the effect when prepared in saline.

FIG. 9 is a diagram illustrating the design of the peritoneal and vesical catheters used in the in vivo mini-pig urodynamic studies.

FIG. 10 shows the effects on intravesical pressure, intravesical voiding pressure, vesico urethral junction (VUJ) pressure, VUJ threshold voiding pressure and VUJ voiding pressure of filling the bladder with saline.

FIG. 11 shows the effects on intravesical pressure, intravesical voiding pressure, vesico urethral junction (VUJ) pressure, VUJ threshold voiding pressure and VUJ voiding pressure of filling the bladder with saline after bolus administration of the compound of formula (Ia).

FIG. 12 shows the effects on arterial pressure during urodynamic measurements in the absence (control) and presence of the compound of formula (Ia).

FIG. 13 is a graph showing the effects of the compound of formula (Ia) on various urodynamic and cardiovascular parameters in a landrace pig.

FIG. 14 is a diagram illustrating the in vitro skin permeation test cells.

FIG. 15 is a graph showing the skin permeation levels of the compound of formula (Ia) dissolved in Eutanol G™.

FIG. 16 is a graph showing the skin permeation levels of the compound of formula (Ia) dissolved in toluene:DMSO (80:20, v/v).

FIG. 17 is a graph showing the skin permeation levels of the compound of formula (Ia) dissolved in isopropylmyristate:Pharmasolve™ (80:20,v/v).

FIG. 18 is a graph showing the mean skin permeation levels of the compound of formula (Ia) for all three test solutions.

### DETAILED DESCRIPTION OF THE INVENTION

The present invention relates to the compound of formula (I) and therapeutic uses of this compound, for example, in the treatment or prophylaxis of urinary incontinence.

1R,2S-Methoxamine is known as a possible treatment for faecal incontinence from WO 03/055474. In experimental studies in which systemic 1R,2S-methoxamine reached appreciable levels, it was surprisingly found by the present inventors that a side-effect of 1R,2S-methoxamine was to produce difficulty in micturition in subjects with an otherwise healthy bladder. It was considered that the difficulty in micturition was caused by 1R,2S-methoxamine having an effect on the bladder and/or urethra. However, it was also observed that 1R,2S-methoxamine produced unwanted systemic side effects such as elevation of blood pressure, thus making 1R,2S-methoxamine unsuitable as a medicament for this indication.

The present inventors have also found that 1R,2S-methoxamine (formula Ib) is effective in inducing contraction of pig bladder neck muscle in vitro, when administered alone. The compound of formula (I) wherein R¹ is a group such that the compound of formula (I) is a prodrug of 1R,2S-methoxamine that is converted within the kidney tubules into its active form or an active metabolite thereof is effective in inducing contraction of pig bladder neck muscle in vitro, when administered in combination with an enzyme suitable for cleaving the R¹ group, and optionally a carboxy group acceptor. For example, the compound of formula (Ia) is effective in inducing contraction of pig bladder neck muscle in vitro, when administered in combination with gamma glutamyl transferase and the acceptor glycylglycine.

The present inventors have also found that the compound of formula (I) is effective in elevating vesicourethral junction (VUJ) baseline pressure, VUJ threshold pressure and VUJ voiding pressure without impeding urinary flow rate or compromising voiding efficiency, when administered in an in vivo pig urodynamic model. In addition, the present inventors have found that the compound of formula (I) produces minimal elevation in arterial pressure when administered in an in vivo pig urodynamic model.

Accordingly, the present invention provides the compound of formula (I) for use as a medicament, in particular for use in the treatment or prophylaxis of urinary incontinence.

The invention also provides the use of a compound of formula (I) as defined above or a pharmaceutically acceptable solvate or salt thereof, including a solvate of such a salt, for the manufacture of a medicament for the treatment or prophylaxis of urinary incontinence.

The invention also provides a method of treating urinary incontinence, which comprises administering a therapeutically effective amount of compound of formula (I) as defined above or a pharmaceutically acceptable solvate or salt thereof, including a solvate of such a salt, to a mammal in need of said treatment.

Incontinence may result from loss of tone of muscles in the bladder neck, urinary tract, vesicular or urethral sphincter; accordingly the invention finds use in increasing tone of smooth muscle in the bladder neck, urinary tract, vesicular or urethral sphincter.

As mentioned above, R¹ is a group such that the compound of formula (I) is a prodrug of 1R,2S-methoxamine that is converted within the kidney tubules into its active form or an active metabolite thereof. That is to say that preferably the compound has very low activity in the body and that group R¹ is cleavable from the molecule to generate a biologically active form. More preferably the R¹ group is linked by an amide bond to the amino group of 1R,2S-methoxamine. Therefore, preferred R¹ groups have a carbonyl group at the attachment terminus. For example, R¹ may be a substituted alkyl group terminating in a carbonyl group, i.e. —C(═O). Such an alkyl group may be substituted with an amine or a carboxylic acid group, for example R¹ may be CO₂H—CHNH₂—(CH₂)ₙ—C(═O), where n is 0-5, preferably 1-3, most preferably n is 2. Most preferably, the R¹ group is a group which is capable of being cleaved by gamma glutamyl transferase. For example, the R¹ group may be gamma L-glutamic acid. A preferred compound of formula (I) is the compound of formula (Ia).

In particular, the present invention provides a compound of formula (Ia) or a pharmaceutically acceptable solvate or salt thereof, including a solvate of such a salt,

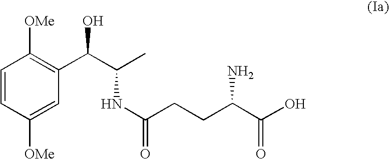

The compound of formula (Ia) is (S)-2-Amino-4-[(1S,2R)-2-(2,5-dimethoxyphenyl)-2-hydroxy-1-methyl-ethylcarbamoyl]butyric acid, also known as gamma-L-glutamyl-1R,2S-methoxamine.

The compound of formula (Ia) may be used as a medicament. The compound of formula (Ia) is a prodrug for 1R,2S-methoxamine. The compound accordingly has use in the treatment or prophylaxis of urinary incontinence.

The subject to be treated according to the present invention is typically a mammal. The mammal is generally a human but may be a commercially reared animal or a companion animal.

The compound of formula (I) may be used as a medicament for the treatment or prophylaxis of any form of urinary incontinence including urge incontinence, reflex incontinence, overflow incontinence, neurogenic incontinence, post-prostatectomy incontinence, transient incontinence and stress or load incontinence. The compound of formula (I) may be used as a medicament for the treatment or prophylaxis of mixed incontinence. The compound of formula (I) finds particular application as a medicament for the treatment or prophylaxis of stress incontinence.

A compound which, upon administration to the recipient is inactive in its administered state but is capable of providing (directly or indirectly) active 1R,2S-methoxamine or an active metabolite thereof, is known as a 1R,2S-methoxamine “prodrug”. A prodrug is converted within the body, into its active form that has medical effects. Pharmaceutically acceptable prodrugs are described in T. Higuchi and V. Stella, Prodrugs as Novel Delivery Systems, Vol. 14 of the A. C. S. Symposium Series; and in Edward B. Roche, ed., Bioreversible Carriers in Drug Design, American Pharmaceutical Association and Pergamon Press, 1987, both of which are incorporated herein by reference.

One example of how a prodrug may be converted within the body into an active form or an active metabolite or residue thereof is by cleavage. It is postulated that the prodrug compounds of the present invention are selectively activated in the kidney due to the presence there of particular enzymes that can cleave the prodrug molecule. Although such enzymes are also found in the liver, significant cleavage of the prodrug compounds in the liver is avoided by selecting an administration route that avoids first-pass metabolism.

An example of a prodrug of 1R,2S-methoxamine is the compound of formula (Ia), that is to say the compound of formula (I) in which R¹ is gamma-L-glutamic acid. The kidney contains the enzyme gamma glutamyl transferase (GGT), which cleaves a peptide bond between glutamic acid and an amine. GGT is found in high levels in the kidney and the liver, although, as noted above, significant cleavage by liver GGT can be avoided by selecting an administration route that avoids first-pass metabolism, e.g. intravenous injection. Thus cleavage of the prodrug when appropriately administered to the body occurs almost exclusively in the kidney.

It is postulated that the glutamic acid-linked agent circulates in the blood until it reaches the kidney, where the peptide bond is cleaved, releasing the agent, which passes with the urine down the ureter, into the bladder and subsequently into the urethra. The agent is thereby selectively active in the urinary system and bladder, and systemic side effects are thus reduced. For example, the typical side effects caused by the muscarinic or anticholinergic agents, for example, dry mouth, visual disturbances, constipation and tachychardia are reduced, and the possibility of using the anticholinergic agents for treatment of patients having prostatic hypertrophy and glaucoma is increased. The invention therefore preferably fulfils the need for agents that are effective in treating urinary incontinence without the side effects of the anticholinergic and muscarinic agents currently in use.

For use in treatment the compound of formula (I) may be used as such, that is to say, in the form of the free base, or in the form of a pharmaceutically acceptable solvate or salt thereof, including a solvate of such a salt. Unless specified otherwise, the term “the compound of formula (I)” as used below includes the free base and pharmaceutically acceptable solvates or salts thereof, including solvates of such salts thereof. When amounts or percentages of the compound of formula (I) or a solvate or salt thereof, or a solvate of such a salt, are given, the amount or concentration of a solvate or salt, or solvate of such a salt, is preferably calculated on the basis of amount or concentration of the compound of formula (I).

Salts of the compound of formula (I) are, for example, salts with acids, including acid addition salts. Examples of salts are those with hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, ethanesulphonic acid, p-toluenesulphonic acid, benzenesulphonic acid, isethionic or camphoric acid.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

The amount of the compound of formula (I) which is required to achieve a therapeutic effect will, of course, vary with the route of administration, the subject under treatment, and the form of urinary incontinence being treated. In clinical treatment, it is generally preferable to use the lowest dose that achieves the desired effect.

The compound of the invention may be administered at a dose of from 0.1 to 1500 mg/kg per day, preferably 0.1 to 500 mg/kg per day. Unit dose forms may conveniently contain an amount of compound of the invention which is effective at such dosage or as a multiple of the same, for example units containing 5 mg to 500 mg, usually around 10 mg to 200 mg.

The compound of the present invention may be administered one or more times per day, for example, two or three times per day, or even more often, for example, four or five times per day.

While it is possible for the active ingredient to be administered alone, it is preferable for it to be present in a pharmaceutical formulation or composition. Accordingly, the invention provides a pharmaceutical formulation comprising a compound of formula (I) as defined above, or a pharmaceutically acceptable solvate or salt thereof, including a solvate of such a salt, in admixture or conjunction with a pharmaceutically suitable carrier or excipient. Pharmaceutical compositions of the invention may take the form of a pharmaceutical formulation as described below.

The pharmaceutical formulations according to the invention include those suitable for parenteral (including subcutaneous, intradermal, intramuscular, intravenous, and intraarticular), inhalation (including fine particle dusts or mists which may be generated by means of various types of metered dose pressurized aerosols, nebulizers or insufflators), topical (including dermal, buccal, and sublingual) and rectal administration, although the most suitable route may depend upon, for example, the nature and stage of the condition and disorder of the recipient.

The compound of formula (I) or a pharmaceutical composition or formulation thereof is preferably administered by a route other than oral administration when used as a medicament for the treatment or prophylaxis of urinary incontinence.

A pharmaceutical composition of the invention may be, for example, in a form suitable for topical administration to the skin, for example, a gel, cream, ointment, paste, foam or adhesive patch. For female patients, the composition may be in a form suitable for intravaginal administration.

A pharmaceutical composition of the invention may be, for example, in a form suitable for rectal administration, for example, a suppository. A sustained release suppository may, for example, be suitable.

A pharmaceutical composition of the invention may be, for example, in a form suitable for transdermal administration. A pharmaceutical composition suitable for transdermal administration may comprise an adjuvant that enhances the transdermal delivery of the compound of formula (I). Any adjuvant that enhances the transdermal delivery of the compound of formula (I) may be used in the composition of the invention regardless of the way in which such enhancement is achieved.

Suitable adjuvants include certain pharmaceutically acceptable materials that have been used as skin permeation enhancers or solubilizers in transdermal drug delivery systems. Exemplary materials include C₈-C₃₆ fatty acids such as isostearic acid, octanoic acid, and oleic acid; C₈-C₃₆ fatty alcohols such as oleyl alcohol and lauryl alcohol; lower alkyl esters of C₈-C₃₆ fatty acids such as ethyl oleate, isopropyl myristate, butyl stearate, and methyl laurate; di (lower) alkyl esters of C₆-C₈ diacids such as diisopropyl adipate; monoglycerides of C₈-C₃₆ fatty acids such as glyceryl monolaurate; tetraglycol (tetrahydrofurfuryl alcohol polyethylene glycol ether); tetraethylene glycol (ethanol, 2,2′ (oxybis (ethylenoxy)) diglycol); C₆-C₃₆ alkyl pyrrolidone carboxylates; polyethylene glycol; propylene glycol; 2-(2-ethoxyethoxy) ethanol; diethylene glycol monomethyl ether; N,N-dimethyldodecylamine N-oxide; dimethylsulfoxide; and combinations of the foregoing.

Alkylaryl ethers of polyethylene oxide, polyethylene oxide monomethyl ethers, and polyethylene oxide dimethyl ethers are also suitable, as are solubilizers such as glycerol and N-methyl pyrrolidone. The terpenes are another useful class of softeners, including pinene, d-limonene, carene, terpineol, terpinen-4-ol, carveol, carvone, pulegone, piperitone, menthone, menthol, neomenthol, thymol, camphor, borneol, citral, ionone, and cineol, alone or in any combination.

A particularly preferred adjuvant is a mixture of isopropylmyristate (IPM) as available from Advance Scientific & Chemical, Inc., USA and Pharmasolve™ (N-methylpyrrolidone, as available from International Speciality Products, Wayne, N.J., USA) 80:20 (v/v). An investigation of the percutaneous absorption of a saturated solution of a compound of formula 1a dissolved in a mixture of isopropylmyristate (IPM) and Pharmasolve™ 80:20 (v/v) is shown in Example 6. The results of the investigation demonstrate an enhancement factor of 13.84 for percutaneous absorption of a compound of formula (Ia) dissolved in IPM/Pharmasolve compared with a control solution of a compound of formula (Ia).

A pharmaceutical composition may be for subcutaneous administration. Some compositions, for example, subcutaneous depot preparations and adhesive patches may provide delayed or sustained release.

A pharmaceutical composition of the present invention may be in unit dosage form. For administration by injection or infusion unit dosage forms include, for example, vials and ampoules. Unit dosage forms for topical administration to the skin include blister packs or sachets, each blister or sachet containing a unit dose of, for example, a gel, cream or ointment, for example, as described above. A metered dosing device may be provided, for example, a pump device, for dosing a predetermined volume of a topical composition, for example, a cream, ointment or gel. A preparation may provide sustained release, for a depot preparation or an adhesive patch.

Pharmaceutical compositions as described above may also comprise one or more further active ingredients in addition to the compound of formula (I), for example, a further active ingredient with efficacy in the treatment of urinary incontinence.

Pharmaceutical compositions of the various types described above and other compositions suitable for the routes of administration described above are known, as are formulations for such compositions and methods for their preparation. The literature of the art includes handbooks, for example, Remington's Pharmaceutical Sciences by EW Martin. Reviews and literature articles describe both standard and more sophisticated formulations and devices, for example, various types of adhesive patches.

Methoxamine (2-amino-1-(2,5-dimethoxyphenyl)-1-propanol) is currently used clinically as a pressor agent and as a vasoconstrictor agent. Methoxamine has two chiral centres and hence has four stereoisomers. It is used clinically in the form of a mixture of isomers.

WO 03/055474 describes the synthesis of 1R,2S-methoxamine and its characterization by nuclear magnetic resonance (NMR) spectroscopy and single crystal X-ray diffractometry. It also describes the use of 1R,2S-methoxamine in the treatment of faecal incontinence.

The present invention provides a process suitable for the production of the compound of formula (I), wherein R¹ is a group such that the compound of formula (I) is a prodrug of 1R,2S-methoxamine that is converted within the kidney tubules into its active form or an active metabolite thereof, which comprises
reacting 1R,2S-methoxamine with a group of the formula R¹—OH, in the presence of a suitable coupling agent and optionally in the presence of a base, wherein R¹ is as defined above.

Suitable coupling agents include EDCI, HOBT, BOP, PyBOP, HATU and other peptide coupling agents known to the person skilled in the art. Suitable bases include amines, for example alkylamines, for example diisopropylethylamine, triethylamine, pyridine, 2,6-lutidine and 1-methylmorpholine or inorganic bases, for example potassium carbonate, potassium tert-butoxide, sodium carbonate. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The present invention also provides a process suitable for the production of the compound of formula (I), wherein R¹ is a group such that the compound of formula (I) is a prodrug of 1R,2S-methoxamine that is converted within the kidney tubules into its active form or an active metabolite thereof, which comprises
reacting 1R,2S-methoxamine with a group of the formula R¹-L, optionally in the presence of a base, wherein R¹ is as defined above and L is a suitable leaving group.

Examples of suitable leaving groups L include halogen, C₁₋₄alkyl sulphonate esters, C₅₋₁₀aryl sulphonate esters or C₅₋₁₀ar-C₁₋₄alkyl sulphonate esters, for example, a chloride, a bromide, a methylsulfonyl or a toluenesulfonyl group. Suitable bases include amines, for example alkylamines, for example diisopropylethylamine, triethylamine, pyridine, 2,6-lutidine and 1-methylmorpholine or inorganic bases, for example potassium carbonate, potassium tert-butoxide, sodium carbonate. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The present invention provides a process suitable for the production of the compound of formula (Ia), which comprises
reacting a compound of formula (II) 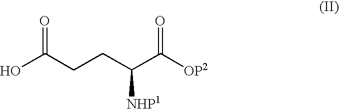
   wherein P¹ and P² are suitable protecting groups
   with 1R,2S-methoxamine, in the presence of a suitable base and optionally in the presence of a one or more suitable coupling agents.

Suitable bases include amines, for example alkylamines, for example diisopropylethylamine, triethylamine, pyridine, 2,6-lutidine and 1-methylmorpholine or inorganic bases, for example potassium carbonate, potassium tert-butoxide, sodium carbonate. Suitable coupling agents include EDCI, HOBT, BOP, PyBOP, HATU and other peptide coupling agents known to the person skilled in the alt. The reaction mixture is stirred at room temperature, or heated until the starting materials have been consumed. The protecting groups P¹ and P² may be removed after the reaction in a single step or in separate steps. The reaction may be carried out with protecting groups other than P¹ and P² present and those protecting groups may be removed after the reaction. Suitable protecting groups are known to the person skilled in the art (see T. W. Greene, “Protective Groups in Organic Synthesis”, 3^{rd} Edition, New York, 1999). Preferably P¹ is benzyl carbamate. Preferably P² is benzyl.

The resulting compound of formula (I) may be converted into a salt thereof, for example, a salt with an acid, for example, a salt as described above, for example, by reaction with an acid. The salt may optionally be isolated as a solvate.

The compound of formula (I) may alternatively be isolated as a solvate.

The invention will now be illustrated by the following Examples, which do not in any way limit the scope of the invention.

### EXAMPLES

### Abbreviations

EDCI 1-(3-Dimethylaminopropyl)-3-ethylcarbodiimide hydrochloride
HOBT 1-Hydroxybenzotriazole
BOP 1-benzotriazolyoxytris(dimethylamino)phosphonium hexafluorophosphate (Castro's Reagent)
PyBOP 1-benzotriazolyoxytris(pyrrolidino)phosphonium hexafluorophosphate
HATU O-(7-azabenzotriazol-1-yl)-1,1,3,3-tetramethyluronium hexafluorophosphate

### Example 1

### Synthesis of the compound of formula (Ia)—(S)-2-Amino-4-[(1S,2R)-2-(2,5-dimethoxyphenyl)-2-hydroxy-1-methyl-ethylcarbamoyl]butyric acid (gamma-L-glutamyl-1R,2S-methoxamine)

Step 1

(1R,2S)-2-amino-1-(2,5-dimethoxyphenyl)propan-1-ol (1R,2S-methoxamine, 16.15 mmol, 4 g), (S)-2-benzyloxycarbonylamino-4-carbamoylbutyric acid benzyl ester (13.5 mmol, 5 g) and EDCI (14.85 mmol, 2.85 g) were suspended in acetonitrile (100 ml) and sufficient water was added dropwise to allow for complete dissolution of all reagents. The reaction mixture was stirred at room temperature for 48 h, then evaporated under a reduced pressure. The residue was purified by column chromatography (EtOAc:hexanes=1:1) to afford (S)-2-benzyloxycarbonylamino-4-[(1S,2R)-2-(2,5-dimethoxyphenyl)-2-hydroxy-1-methyl-ethylcarbamoyl]butyric acid benzyl ester (2.3 g, 30%) as a colourless oil: MS (+EI) 565 [M+1]⁺.

Step 2

To (S)-2-benzyloxycarbonylamino-4-[(1S,2R)-2-(2,5-dimethoxyphenyl)-2-hydroxy-1-methyl-ethylcarbamoyl]butyric acid benzyl ester (4.07 mmol, 2.3 g) in methanol (80 ml) was added 10% Pd—C (0.41 mmol, 0.44 g). The flask was fitted with a balloon containing H₂, and the reaction mixture was stirred at room temperature overnight. The reaction mixture was filtered through Celite, which was washed with methanol. Concentration under a reduced pressure gave (2S)-2-Amino-4-[(1S,2R)-2-(2,5-dimethoxyphenyl)-2-hydroxy-1-methyl-ethylcarbamoyl]butyric acid {gamma-L-glutamyl-1R,2S-methoxamine} (1.0 g, 72% yield) as a solid: MS (+EI) 341 [M+1]⁺, 323 [M-OH].

### Comparative Example 2

### Synthesis of the compound of formula (III)—(S)-4-Amino-4-[(1S,2R)-2-(2,5-dimethoxyphenyl)-2-hydroxy-1-methyl-ethylcarbamoyl]butyric acid (alpha-L-glutamyl-1R,2S-methoxamine)

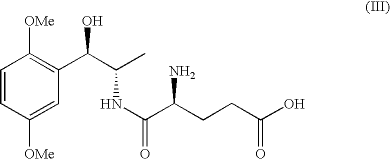 Step 1

(1R,2S)-2-amino-1-(2,5-dimethoxyphenyl)propan-1-ol (1R,2S-methoxamine, 16.15 mmol, 4 g), (S)-4-benzyloxycarbonylamino-4-carbamoylbutyric acid benzyl ester (13.5 mmol, 5 g) and EDCI (14.85 mmol, 2.85 g) were suspended in acetonitrile (100 ml) and sufficient water was added dropwise to allow for complete dissolution of all reagents. The reaction mixture was stirred at room temperature for 7 days, then evaporated under a reduced pressure. The residue was purified by column chromatography (EtOAc:hexanes=1:1) to afford (S)-4-benzyloxycarbonylamino-4-[(1S,2R)-2-(2,5-dimethoxyphenyl)-2-hydroxy-1-methyl-ethylcarbamoyl]butyric acid benzyl ester (1.9, 25%) as a colourless oil: MS (+EI) 565 [M+1]⁺.

Step 2

To (S)-2-benzyloxycarbonylamino-4-[(1S,2R)-2-(2,5-dimethoxyphenyl)-2-hydroxy-1-methyl-ethylcarbamoyl]butyric acid benzyl ester (3.37 mmol, 1.9 g) in methanol (38 ml) was added 10% Pd—C (0.19 g). The flask was fitted with a balloon containing H₂, and the reaction mixture was stirred at room temperature overnight. The reaction mixture was filtered through Celite, which was washed with methanol. Concentration under a reduced pressure gave (S)-4-amino-4-[(1S,2R)-2-(2,5-dimethoxyphenyl)-2-hydroxy-1-methyl-ethylcarbamoyl]butyric acid {alpha-L-glutamyl-1R,2S-methoxamine} (1.1 g, 96% yield) as a solid: MS (+EI) 341 [M+1]⁺, 323 [M-OH].

### Example 3

### In Vitro Study of the Compound of Formula (Ia) and the Compound of Formula (III) on Pig Bladder Neck Muscle

a) Functional Assay

Method

Sample Collections and Preparation

Samples of female white landrace pig bladder and urethra were obtained from freshly slaughtered pigs at the local abattoir. The pigs were killed by exsanguination after initial stunning by electric shock. The anus, rectum, bladder and urethra were then excised en bloc during evisceration and immediately transferred into pre oxygenated Krebs' solution (1 mmol/l: NaCl 120, KCl 5.9 NaHCO₃ 15.4, CaCl₂ 2.5, MgCl₂ 1.2, glucose 11.5) and transported to the laboratory, where they were stored in the refrigerator (at 4° C.) until ready to be processed.

The urinary bladder and urethra were removed from the above samples and pinned down dorsally onto a dissecting dish where the bladder was cut open to expose the trigone, and the urethra was dissected free of the bladder at the apex of the trigone.

The location of the vesicourethral junction sphincter was then identified. The bladders were inflated via a transurethral catheter with 200 ml. fluid. The fluid was then gently squeeze towards the outlet, until a sharp narrowing of the bladder base becomes apparent (Dass, 1997; Dass et al., 2001). Tissue sections from this region were then denuded of urothelium, and dissected transversely into a number of muscle strips.

Tissue Dissection

Dissection was carried out in the silicon elastomer coated base of a dissecting Petri-dish (containing oxygen saturated Krebs' solution which was continually replenished to ensure constant oxygenation of the tissue) aided by a dissecting microscope.

Mounting and Equilibration of Strips

Muscle strips measuring about 5-6 mm by 2-3 mm with an average weight of 7 mg were dissected, fine (5/0) silk threads were tied at each end of the strips and each strip mounted vertically in a 0.2 ml capacity, perspex organ bath between two recessed platinum electrodes through which electrical field stimulation (EFS) could be delivered. Strips were constantly superfused with Krebs' solution (37° C.) at a rate of 1.5 ml 1 min. The Krebs' solutions were gassed with 97% oxygen 3% carbon dioxide. Drugs were delivered by substituting drug containing solutions of the desired concentration for Krebs' solutions.

Apparatus

The apparatus comprised of six organ baths in parallel allowing the study of six strips simultaneously as described by Brading and Sibley (1983). Each channel was calibrated with a 1 g weight attached to the transducer before each experiment, and tissues were tensioned to 1 g after mounting. Isomeric tension generated by the strips was measured using isomeric force displacement transducers, Pioden Dynamometer UFI transducers (Pioden Controls Ltd, Canterbury, Kent) or AD Instruments MLT050/D force transducers, amplified (Harvard Transducer/Amplifier, Harvard Apparatus Ltd, Edenbridge, Kent) and analyzed using MacLab Data Acquisition system (AD Instruments, Sydney, Australia) or AD Instruments OCTAL Bridge Amplifier connected to Power Lab/8SP (AD Instruments, Sydney, Australia) and Chart v3.6 software (Macintosh) or Chart v5 (PC).

Dose Response Curve Protocol

Dose response curves for the various compounds were constructed by testing one drug on a pair of strip in each experiment. The preparations were subjected to a sequence of successive 10 s (@1.5 ml/s) applications of increasing doses of drugs with intervening washout periods.

Drugs and Reagents

1R,2S-methoxamine hydrochloride, the compound of formula (Ia) and the compound of formula (III) were made available by Norgine International Research, Middlesex.

Data Analysis

Responses were reported as either grams of tension or % maximum force and given as mean values ±SEM. EC₅₀ values were used as a measure of potency. The EC₅₀ values were measured from the graph of dose response curve using the kaleidograph graphical software applications. Results were assessed using statistical analysis software, StatView 5.0 (SAS Institute Inc.) using ANOVA followed by Fisher's test. Significant difference between data was assumed with p<0.05.

Results

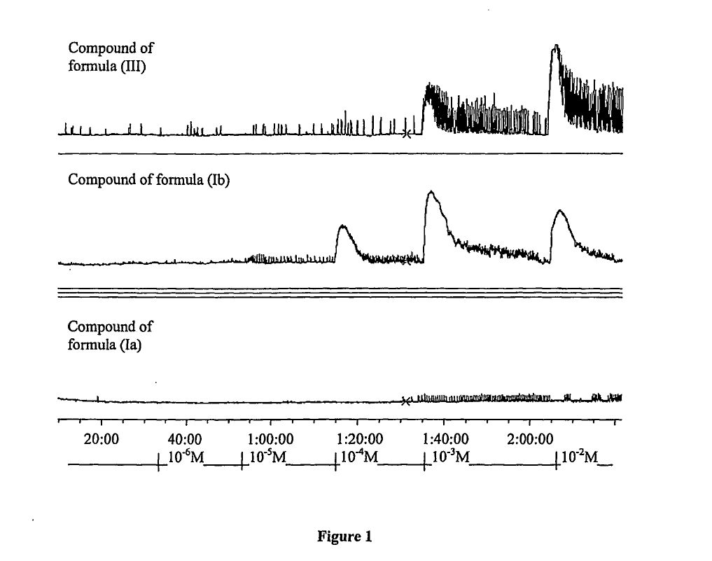

Representative traces of isolated pig vesicourethral junction sphincter muscle strip responses to the compound of formula (III), 1R,2S-methoxamine, and the compound of formula (Ia) are shown in FIG. 1. 1R,2S-Methoxamine caused a pronounced well defined contractile response of the pig vesicourethral junction sphincter muscle (FIG. 1, middle trace). The compound of formula (III), produced by chemical synthesis as described in comparative Example 2, also causes a well defined contractile response of the pig vesicourethral junction sphincter muscle (FIG. 1, top trace). The compound of formula (Ia), produced by chemical synthesis as described in Example 1, caused no significant contraction of the pig vesicourethral junction sphincter muscle (FIG. 1, bottom trace).

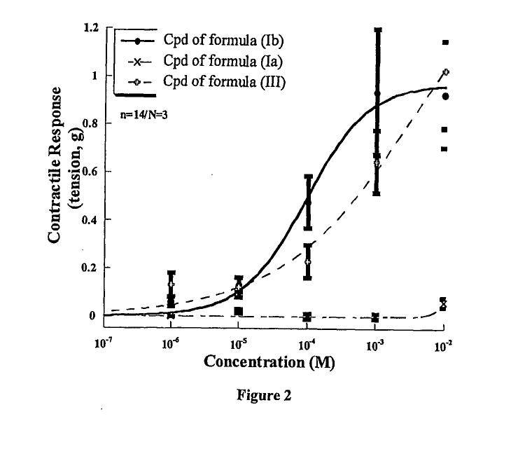

Graphs of contractile response of pig vesicourethral junction sphincter muscle to increasing doses of either 1R,2S-methoxamine, the compound of formula (Ia) or the compound of formula (III) are shown in FIGS. 2a and 2b. 1R,2S-methoxamine induced a maximum response of 1.12 g±0.26 g at a concentration of 10⁻³M (FIG. 2a). The compound of formula (III) also exhibited some bladder activity but a maximum response of 1.33 g±0.25 g was only observed at 10⁻²M whereas the compound of formula (Ia) failed to evoke any appreciable response over the dose range (10⁻⁶M-10⁻²M) tested (FIG. 2a). Both 1R,2S-methoxamine and the compound of formula (III) generated a typical sigmoid dose response curve as opposed to the flat response observed with the compound of formula (Ia) (FIG. 2b). The relative potencies (EC₅₀) are in the order of 1R,2S-methoxamine (0.0562 mM)> the compound of formula (III) (0.495 mM)> the compound of formula (Ia) (4.64 mM). Therefore the compound of formula (III) retains approximately 10% of the biological activity of 1R,2S-methoxamine.

Conclusions

The compound of formula (Ia) has little or no activity in a standard in vitro muscle strip test (FIG. 1), which indicates that the compound has prodrug status. However, the compound of formula (III), has some activity in a standard in vitro muscle strip test and was found to retain approximately 10% of the biological activity of 1R,2S-methoxamine. Therefore the compound of formula (III) in which glutamic acid is coupled to 1R,2S-methoxamine via the alpha carboxy group is not as suitable as the compound of formula (Ia) in which glutamic acid is coupled to 1R,2S-methoxamine via the gamma carboxy group for use as a pro-drug in the treatment or prophylaxis of urinary incontinence.

b)Enzyme Assay

Method

Validation Phase

The commercially purchased gamma glutamyl transferase (γGT) was validated with the commercial substrate, glutamyl p-nitroanilide (GpNA) and co-factor glyglycine (glygly) and magnesium. The reaction can be characterized by the equation below: 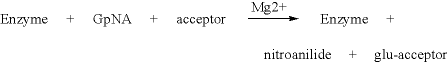
γGT should release nitroanilide from the conjugate GpNA to produced a reaction product with yellowish color which can be measured at the λₘₐₓ405 nm.
a) Stock solutions were prepared as stated below:

In 50 mM Tris HCl buffer pH 8.2 the following stocks were made:

A. Glycylglycine (glygly) (acceptor) 20 mg/ml in Tris HCl buffer

B. MgCl₂ 6.H₂O 3.3 mg/ml in Tris buffer

C. Gamma glutamyl p-Nitroanilide 6 mg/ml in Tris buffer

D. Enzyme stock solution was prepared by dissolving γGT (3000 U) in 10 ml Tris buffer (=300 U/ml) and taking aliquots into 20 μl (=500) stock solution to store at −20° C.

E. Enzyme working solution. Reconstitute 1 aliquot (=6 Units) into 1 ml Tris buffer (=6 units/ml).

b) Enzyme Assay Protocol

GpNA was assayed spectrophotometrically in 96 wells as follow:

In replicate wells the following was pipetted:
60 μl Solution A
60 μl Solution B
60 μl Solution C (=0.36 mg or 1.26μ Moles)

Reaction mixture groups:

| | |
|---|---|
| 1 = Buffer only (200 μl) | Blank |
| 2 = Solution A (60 μl), Solution B (60 μl), | Control |
| Solution C (60 μl), Buffer (20 μl) | |
| 3 = Buffer (180 μl) + Solution E (20 μl) | Control |
| 4 = Solution A (60 μl), Solution B (60 μl), | Test |
| Solution C (60 μl), Solution E (20 μl) | |

To the groups that received 20 μl of solution E (=0.12 units γGT), this solution (E) was only added after 10 minutes of equilibration at 37° C. The plates were then read (@λₘₐₓ 405 nm) immediately at time 0 and thereafter every 5 min after an intervening equilibration period until the plateau were reached.

NB. 1 U cleaves 1 μMole per minute so 0.12 units should cleave 1.26 μMole in 10.5 minutes

Regeneration of 1R,2S-methoxamine from the Compound of Formula (Ia)

Into a clean glass vial (1 ml volume) the following reaction mixtures were pipetted as follows:

100 μl solution A

100 μl solution B

200 μl compound of formula (Ia), 10 mg/ml in Tris buffer (mol. wt. 376, therefore=2 mg compound of formula (Ia)=5.3 μMoles), 100 μl solution E

Reaction mixture groups:

| | |
|---|---|
| 1 = Buffer only (200 μl) | Blank |
| 2 = Solution A (60 μl), Solution B (60 μl), | Control |
| Solution C (60 μl), Buffer (20 μl) | |
| 3 = Buffer (180 μl) + Solution E (20 μl) | Control |
| 4 = Solution A (60 μl), Solution B (60 μl), | Test |
| Solution C (60 μl), Solution E (20 μl) | |

100 μl solution E = 0.6 units should cleave 5.3μ Moles in 8.8 minutes.

Reaction product of groups 1, 2 and 3 were only tested for biological activity after 30 minutes of incubation period at 37° C. while that of group 4 were also tested after, 10, 30 and 60 minutes of incubation period. A 5 h group of which reaction mixture indicated in group four were diluted by two fold after 30 min incubation were also tested for biological activity.

Reagents

Gamma glutamyl transferase, 2-1-18 porcine kidney was purchased from Calzyme Labs San Luis Obispo, Calif. 93401 USA, glyclglycine was obtained from Acros Organics, New Jersey, USA while L-glutamic acid 5-(4-nitroanilide) was purchased from Fluka Chemie Switzerland.

Apparatus

The reaction mixture was incubated in HOT BOX oven, size one incubator made by Gallenpark, England, while the absorbance was measured (@λₘₐₓ 405 nm), using Anthos 2020 UV/VIS spectrophotometer.

Results

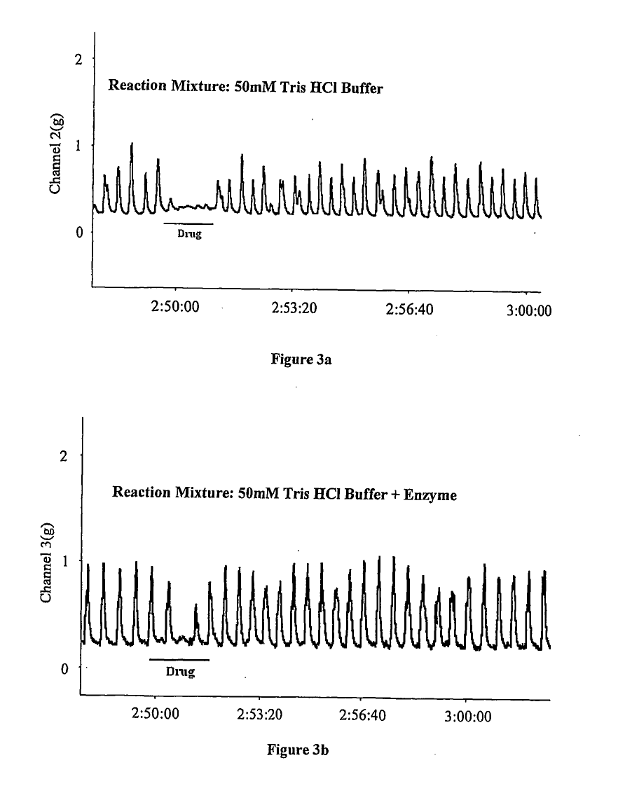

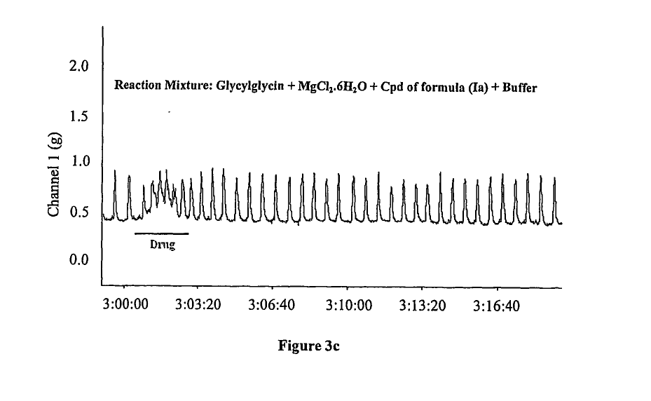

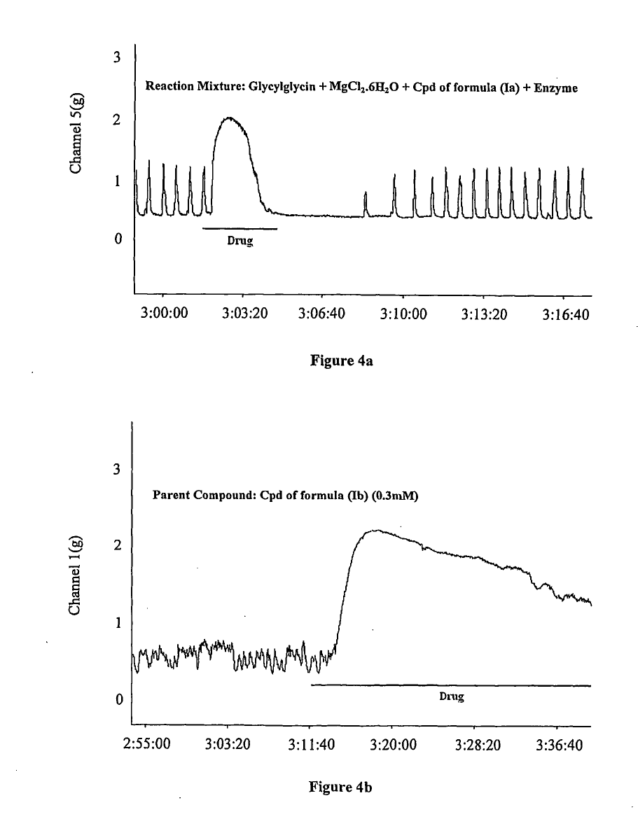

Representative traces of pig vesicourethral junction sphincter muscle response to various enzyme reaction products are shown in FIG. 3 and representative traces of pig proximal sphincter muscle response to various enzyme reaction products are shown in FIG. 4. The enzyme reaction products tested are as follows:
FIG. 3a-50 mM Tris HCl buffer;
FIG. 3b-50 mM Tris HCl buffer, gamma glutamyl transferase.
FIG. 3c-50 mM Tris HCl buffer, MgCl₂.6H₂O, glycylglycine, compound of formula (Ia);
FIG. 4a-50 mM Tris HCl buffer, MgCl₂.6H₂O, glycylglycine, gamma glutamyl transferase, compound of formula (Ia);
FIG. 4b-0.3 mM 1R,2S-methoxamine.

The compound of formula (Ia), administered in conjunction with gamma glutamyl transferase and glycylglycine, caused a pronounced well defined contractile response of the pig bladder neck muscle (FIG. 4a), comparable to that observed with 1R,2S-methoxamine (FIG. 4b).

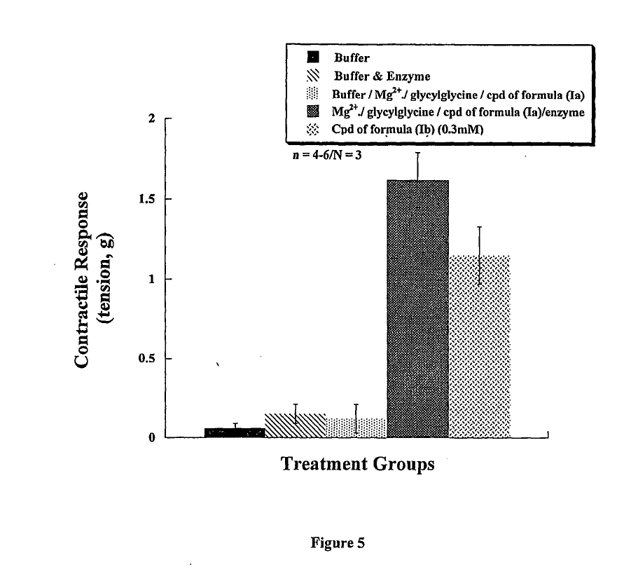

A comparative graph of the contractile response of pig bladder neck muscle under the various reaction conditions described for FIGS. 3a-c and 4a-b is shown in FIG. 5. The graph shows the contractile response of vesicourethral junction sphincter to 1R,2S-methoxamine and the individual products of the reaction between the compound of formula (Ia) and the enzyme gamma glutamyl transferase. Neither the buffer alone (0.06 g±0.03 g) nor the buffer plus enzyme produced any significant activity (0.5 g±0.06 g). The reaction mixture of buffer-Mg²⁺-glycylglycine-compound of formula (Ia) also failed to produce any significant activity (0.12 g±0.09 g). However the mixture of Mg²⁺-glycylglycine-compound of formula (Ia)-enzyme did evoke a robust bladder activity (1.62 g±0.17 g). 1R,2S-methoxamine also evoked a bladder response (1.15 g±0.18 g) but this was 29% less effective than that observed for Mg²⁺-glycylglycine-compound of formula (Ia)-enzyme (1.62 g±0.17 g). The results demonstrate that the compound of formula (Ia) has activity in the assay in the presence of gamma glutamyl transferase. That observation in combination with the observation above that the compound does not have activity on its own strongly suggests that the compound is a pro-drug.

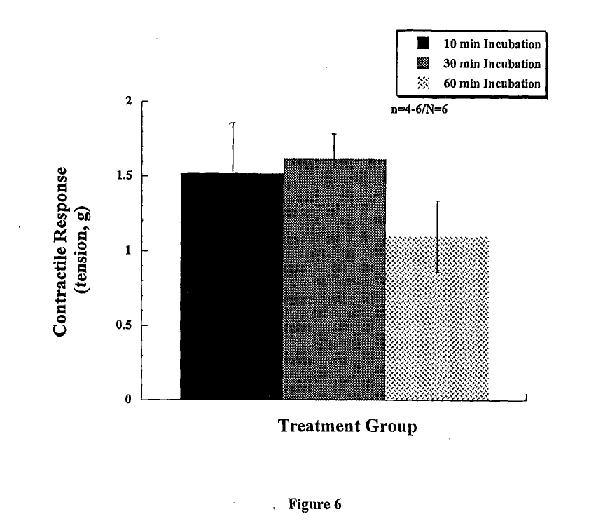

The time-course of the reaction mixture's effect on vesicourethral junction sphincter muscle strips is shown in FIG. 6. The aim was to determine the time at which the reaction with the compound of formula (Ia) produced the most marked effect on the muscle. The efficacy of the reacting mixture was measured at 10, 30 and 60 min after incubation. There was no apparent difference in responses between reaction mixtures incubated for 10 min (1.52 g±0.34 g) and 30 min (1.62 g±0.17 g), however a small reduction in activity was observed at 60 min (1.10 g±0.24 g) incubation.

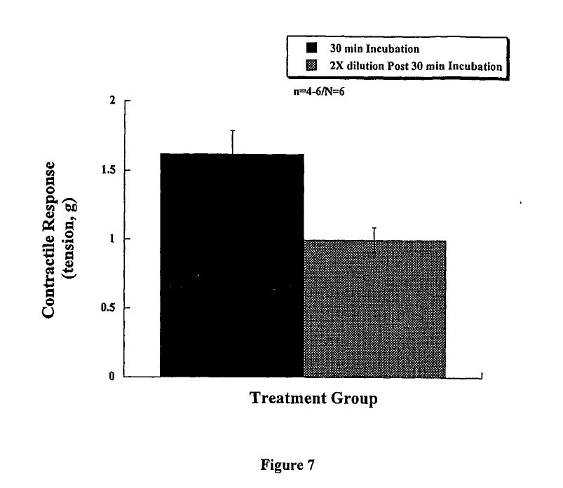

The effect of the compound of formula (Ia) on vesicourethral junction sphincter muscle strips after a 2-fold dilution post 30 min incubation with the enzyme is shown in FIG. 7. Bladder activity of the reaction mixture was reduced by 38% after the 2-fold dilution.

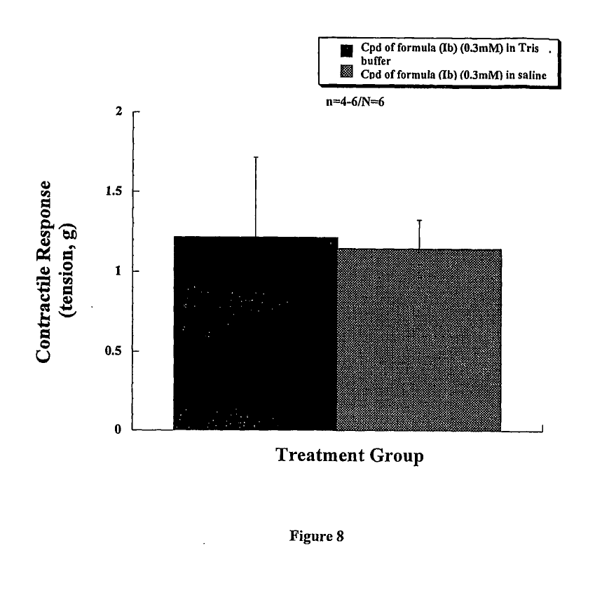

The effect of 1R,2S-methoxamine (0.3 mM), prepared in either tris buffer or saline, on vesicourethral junction sphincter muscle strips is shown in FIG. 8. There was no appreciable difference in response between the compound of formula (Ia) prepared in tris buffer (1.22 g±0.50 g) and that prepared in saline (1.15 g±0.18 g).

### Example 4

### Examination of the Influence Effect of 1R,2S-methoxamine on Micturition

Method

The study was conducted according to the following protocol:

Test Substances:

a) 1% by weight 1R,2S-methoxamine in 4% w/w gel
b) 3% by weight 1R,2S-methoxamine in 4% w/w gel
The 4% w/w gel comprises 4 g of hydroxypropylmethyl cellulose in 96 ml of 0.5M acetate buffer. 1% by weight 1R,2S-methoxamine in 4% w/w gel comprises 1 g of 1R,2S-methoxamine in 99 g of the 4% gel.
   Investigation

In a study for other purposes including 44 subjects, each subject had either the 3% methoxamine gel or the 1% methoxamine gel applied intra-anally. About 1 g of gel was used.

Results

Four subjects reported difficulties in micturition, two who had received the 1% gel, two who had received the 3% gel. The other volunteers did not report experiencing difficulty in micturition.

Conclusions

The administration of a 1% or 3% 1R,2S-methoxamine gel intra-anally affected the urinary system, causing difficulty in micturition.

The effects observed, in particular the difficulty in micturition, demonstrate the utility of 1R,2S-methoxamine or 1R,2S-methoxamine derivatives that may be converted to 1R,2S-methoxamine in the kidney tubules in the treatment of urinary incontinence and other conditions resulting from loss of tone and/or loss of function of any one or more of the bladder neck, the urinary tract and the urinary sphincters.

### Example 5

### In Vivo Study of the Compound of Formula (Ia) on Urodynamic and Cardiovascular parameters in a pig model

Method

Surgical Procedure

The animal model employed in this study was a slight modification of the previously described pig in vivo urodynamic model by Mills (1999). Surgical procedures were performed by Home Office personal licence holders. Female land race pigs of about 60 kg in weight were used for this study.

Anaesthesia

Pigs were initially sedated with ketamine hydrochloride 15 mg/kg (ketaset, Willows Francis Veterinary, Crawley, UK) and midazolam 0.2 mg/kg (Hypnovel, Roche Products Ltd, Welwyn Garden City, UK) through an intravenous central line. Sedation was usually induced 15 to 20 minutes before the induction of general anaesthesia. The induction phase of general anaesthesia was accomplished with 5% halothane (Fluothane, Astra ZenecaLtd, Macclesfield UK) in nitrous oxide 50%/oxygen 50%, delivered from an anaesthetic machine through a conical mask which was held over the snout of the pig. Following the induction phase, the maintenance phase of general anaesthesia was achieved using intravenous 1% propofol at the rate of 200 mg/hr.

Preparation of Catheters

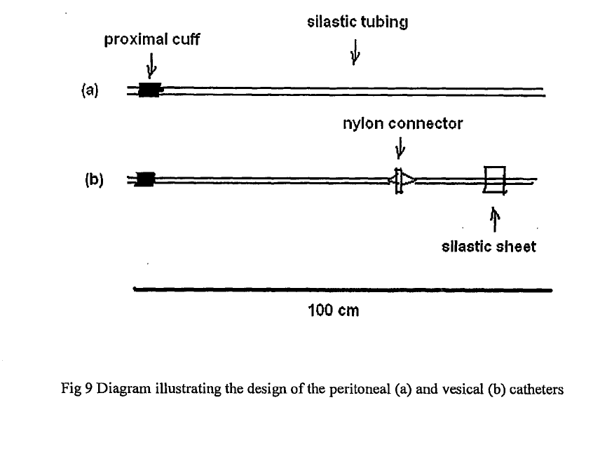

As shown in FIG. 9 Silastic tubing (Bibbly Sterilin Ltd, Stone, Uk) of 1.5 mm bore and 1 mm wall thickness was cut into catheters of approximately 100 cm in length; for each animal, two such catheters were required for vesical lines (one for filling the bladder, the other for measuring intravesical line) and a third one was required for the peritoneal cavity to measure the abdominal pressure. The proximal end of each had a cuff of single valour polyester fabric (Meadox Medicals, Oakland, N.J., USA) fixed with silicone glue (RS components, Corby, UK) approximately 3 cm from the end. The distal end was tapered and had several holes cut in it to help prevent occlusion following implantation. The two vesical catheters were further prepared by bisecting them, 20 cm from the end and then gluing a small piece (2 cm by 1 cm) of silastic sheet approximately half way along the smaller half. These two halves could then be connected using nylon connectors (Microplastics, Coventry, UK).

Implantation of Indwelling Catheters

At the start of surgery, the pig was initially placed in the left lateral position allowing access to the lumbar dorsal region. A stab incision for each catheter was made in the lumbar dorsal midline, as well as a single small right iliac fossa incision. Using a specially designed trocar, the peritoneal catheter and the proximal portions of the vesical catheters were tunneled from the dorsal lumbar incision to the right iliac fossa, such that the cuff at the proximal end finished in a small subcutaneous pocket. Each incision was approximated with two interrupted 2/0 nylon sutures, taking care to avoid occlusion of the catheters. The pig was then turned into a supine position and a lower abdominal incision made. Using a Spencer-Well forceps, the peritoneum and abdominal wall muscles were pierced from inside out and three catheters grabbed and pulled into the peritoneal cavity. The longer peritoneal catheter was placed in the vesico-vaginal pouch. Two purse-string sutures of 2/0 silk were placed in the apex of the bladder, and stab incisions made through the centre of each of these. The distal shorter halves of the vesical lines were introduced into the bladder through these incisions and the purse-strings tied, ensuring that the lumen of each catheter remained patent. Each side of the small pieces of silastic sheet on these catheters was sutured to the serosa of the bladder, in order to provide further security against subsequent line displacement. These distal portions of the two vesical catheters were then connected to the proximal portions using the nylon connectors. The abdominal wound was closed en masse with 1 nylon, followed by 2/0 chromic catgut to fat and interrupted 2/0 nylon to the skin. The right iliac fossa wound was closed in layers with vicryl to internal oblique and external oblique and interrupted 2/0 nylon to the skin. One of the vesical lines was attached to another piece of tubing externally and left on free drainage for 48 hours to allow the bladder to heal.

Urethra and Arterial Blood Vessel Cannulations

Following induction of anaesthesia, the animal was placed in a supine position and covered with a warm water blanket to maintain body temperature. A small incision was then made in the left groin area through which the left femoral artery was identified by palpation. The artery was then cannulated with an arterial 14 Fr cannulae (Abbocath-T, Abbott Ireland, and Sligo, Ireland) which was then connected to a pressure transducer for the measurement of arterial pressure. Vesico-urethral junction cystometry was obtained using a catheter mounted with a microtranducer (Gaeltec LTD, Isle of Skye). To cannulate the urethra, the vagina was opened with a speculum to expose the external urethral orifice which is high up in the anterior wall of the vagina. Having identified the external orifice the catheter was then inserted into the urethra by direct vision, thereafter it was gradually withdrawn until it was positioned in the vesico-urethral junction.

Protocols

Upon instrumentation of the animal and after a stabilization period, three voiding cycles were recorded in the absence and then after bolus administration of the compound of formula (Ia) (1 mg/kg. iv). The dose of the compound of formula (Ia) employed in this study was based on the cardiovascular effect of the compound of formula Ib in mini-pig in vivo studies (Norgine International Research, unpublished). In that study the compound of formula Ib transiently increased systolic and diastolic pressures of mini pigs at 1 mg/kg. i.v. The effect of the compound of formula Ib was at a maximum after fifteen minutes of drug administration, raising systolic and diastolic pressure by 23 and 57% respectively (Norgine International Research, unpublished). These observations in conjunction with the enzyme study were the basis of testing the compound of formula (Ia) at 1 mg/kg., i.v.

For each voiding cycle, the bladder was slowly filled through the intravesicle line with room temperature saline solution at the rate of 48 ml/min until commencement of voiding. During each cycle the following parameters were recorded; intravesical pressure (IVP), deutrusor voiding pressure (DvP), vesicourethral junction threshold voiding pressure (VUJTVP), vesicourethral junction voiding pressure (VUJVP), abdominal pressure (AbP) and the arterial pressure (AP). Other parameters include duration of voiding cycle, that is time taken to complete a micturation cycle (DVC), and the time taken for the voiding to laps (DV). Voided volume (VV) and the residual volume (RV) were also obtained. Bladder capacity (BC) was computed as the sum of VV and RV while the flow rate (FR) is the measure VV per minute. Voiding efficiency was also established, and measured as percentage of VV from the BC. Deutrusor maximum pressure is a measure of the difference between intravesical and abdominal pressure which can be expressed as DvP=IVP−AbP. Data were generated on one animal with each data point representing the mean of three voiding cycles in the absence (control) and after a bolus administration of the compound of formula (Ia).

Results

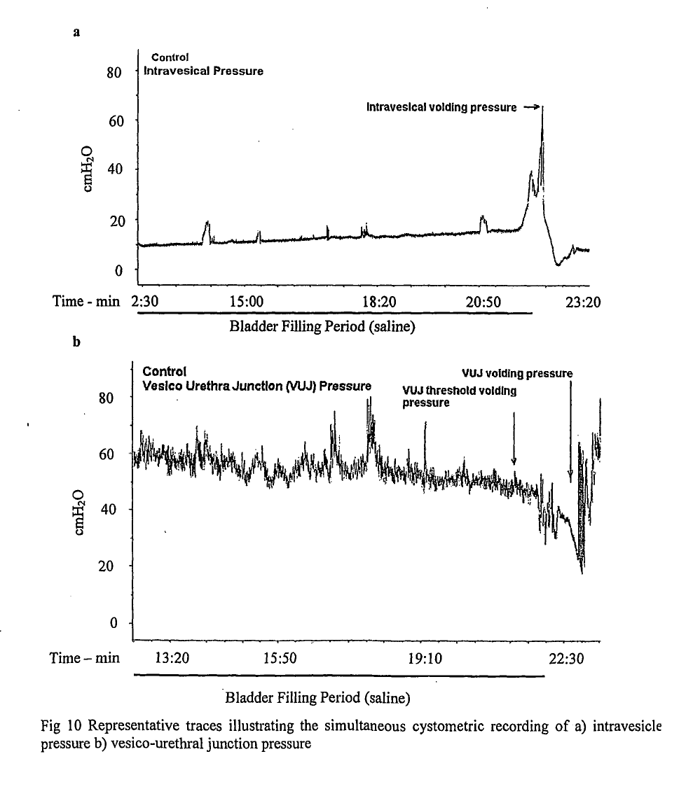

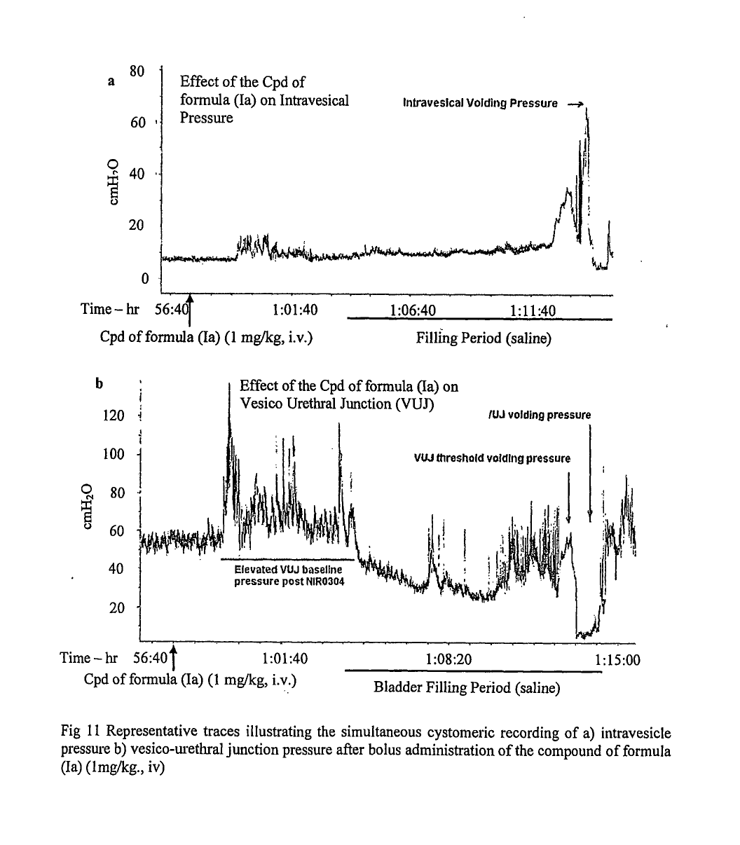

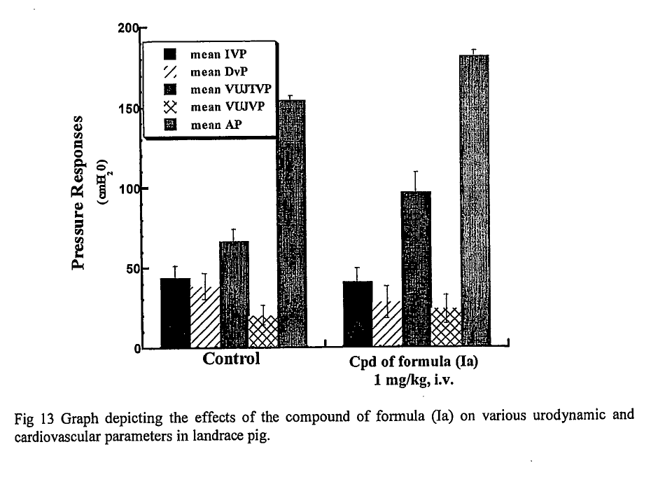

As shown in FIG. 10a, during the filling phase the bladder intravesical pressure gradually rose from the baseline pressure (8.40±0.17 cmH₂O) to a threshold voiding pressure (16.60±1.61 cmH₂O) in the control runs. A similar observation (FIG. 11a) was recorded in the run in the presence of the compound of formula (Ia)—a rise in baseline pressure from 8.53±0.34 cmH₂O to a threshold voiding pressure of 16.23±1.16 cmH₂O. Equally there was no significant difference in the voiding pressure generated during the voiding phase between the control runs (43.9±7.57 cmH₂O) and in the presence of the compound of formula (Ia) (41.1±8.3 cmH₂O) (FIG. 10a and 11a, table 1 and FIG. 13). A gradual rise in deutrusor pressure during filling, which resulted in voiding at the pressure of 38±8.11 cmH₂O for the control and 28±10.11 cmH₂O for the compound of formula (Ia), were also noted (table 1 and FIG. 13). The changed deutrusor voiding pressure represented a 36.5% fall from the control runs to the compound of formula (ha).

In contrast to the benign effect of the compound of formula (Ia) on the bladder pressure parameters, further analysis of the cystometrogram shows that the compound of formula (Ia) did modify the activity of the VUJ. When comparing the control runs (FIG. 10b) with that of the compound of formula (Ia) (FIG. 11b), a distinct picture of transient rise in VUJ baseline pressure was apparent with the compound of formula (Ia) runs. This effect was shown to be accompanied by a more pronounced spontaneous activity that persisted throughout the voiding cycle (FIG. 11b). VUJ threshold pressure produced with the compound of formula (Ia) was about 31% greater than that seen in the control runs. A 15% rise in the threshold VUJ voiding pressure was also observed with the administration of the compound of formula (Ia).

While a 31% reduction in residual volume was observed with the compound of formula (Ia), all the other urodynamic parameters appear to have remained more or less unchanged, the flow rate, duration of voiding and voiding intervals were only slightly changed from the control values by 2.4, 4.1 and −4.7% respectively. Voiding efficiency also appears to remain un-compromised, 90% voiding efficiency was attained with the control runs while 95% was attained under the treatment with the compound of formula (Ia).

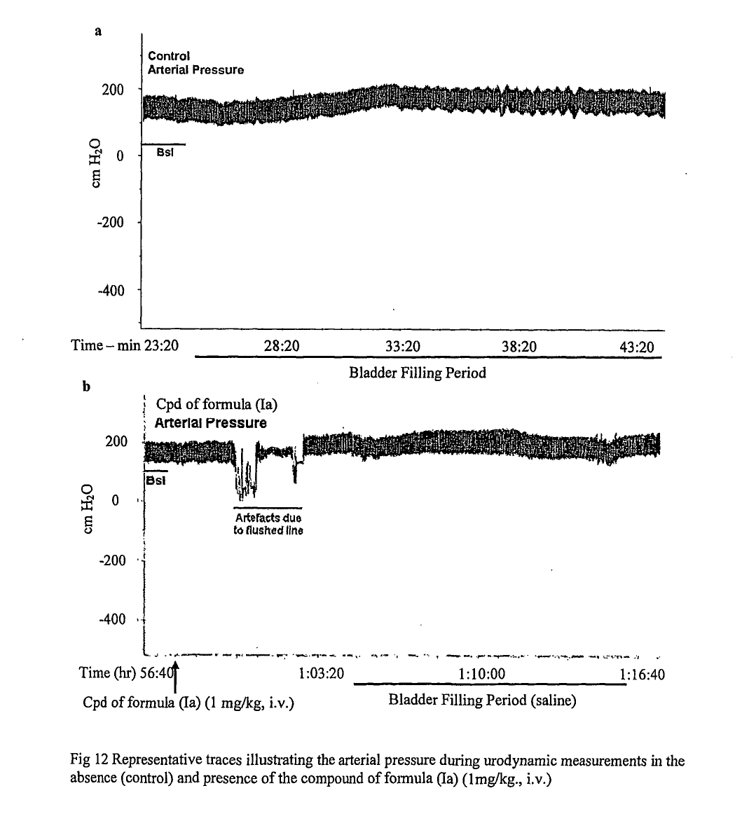

With regard to the arterial pressure effect, the compound of formula (Ia) caused a change of 15% from the control. It should however be noted that soon after drug administration, the arterial line had to be flushed as a result of clotting in the line. This action may have contributed to the increase in blood pressure observed (FIG. 12, table 1 and FIG. 13).

Conclusions

These data demonstrate that the compound of formula (Ia) is capable of elevating vesicourethral junction baseline pressure, threshold pressure and voiding pressure without impeding urinary flow rate or compromising voiding efficiency in an in vivo pig urodynamic model. In addition, while the compound of formula (Ia) did slightly increase arterial pressure, the effects were less marked than those seen with the compound of formula Ib—a 15% increase in arterial pressure was observed with the compound of formula (Ia) (1 mg/kg, i.v.), compared with increases of 23% and 57% in systolic and diastolic pressures respectively which were observed with the compound of formula Ib (1 mg/kg, i.v.). As noted above, the increase in arterial pressure observed with the compound of formula (Ia) may well have been an artifact due to line flushing.

The data therefore indicates the viability of the pro-drug system (the compound of formula (Ia)) in delivering active 1R,2S-methoxamine (the compound of formula Ib) to the bladder, while minimising the unwanted blood pressure side effects seen with the compound of formula Ib.

### Example 6

### Investigation of In Vitro Percutaneous Absorption of Saturated Solutions of the Compound of Formula (Ia) in Excised Human Skin

In this experiment, the skin permeation of the compound of formula (Ia) was investigated.

Method

Excised Human Skin

For all experiments (one run with 9 cells) skin from one female patient was used. The skin was excised full skin from surgery of the thigh, cleaned from adhering fat tissue, stored at −18° C. prior to use but not for longer than 6 months.

Test Design

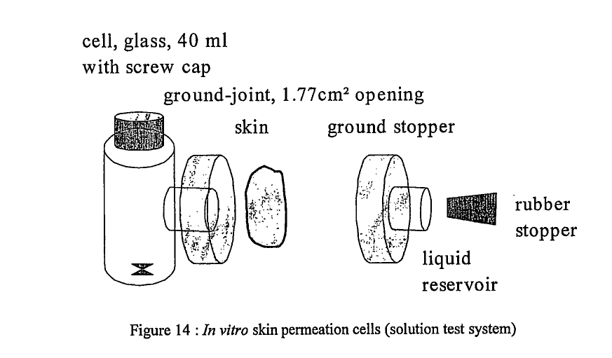

In vitro skin permeation investigation was performed as described in this Example. The cells were mounted as shown in FIG. 14.

Conditions

| | |
|---|---|
| Cell: | Franz cell, modified (s. Pic.1), 35 ml volume, n = 9 |
| Acceptor: | 0.9% NaCl 80% (V/V), PEG 400 20% (V/V), |
| | 0.1% NaN₃ (Water HPLC quality, |
| | degassed), 100 ml |
| Permeation area: | 1.77 cm² |
| Temperature: | 32° C. ± 0.5° C. (water bath) |
| Revolution speed: | 3 (IKA magnetic stirrer); star-shaped fish |
| Sampling volume: | 2.0 ml |
| Replacing volume: | 2.0 ml |
| Sampling interval: | 6, 12, 24, 48, 72 hours |
| Test procedure: | For the test-solution 3 ml of the donor solution |
| | is applied by a pipette into the solution device |
| | and occluded by a screw. This is the start of |
| | the test. |

Products
(For calculation of values please refer to 4.6)

| | |
|---|---|
| Test solution #1: | Solution of the compound of formula (Ia) in Eutanol G ™. |
| | Solution was prepared the day before study by adding 551.5 mg |
| | of the compound of formula (Ia) to 25 ml of Eutanol G ™ (22.1 mg/ml |
| | saturised solution). Concentration was 0.030 mg/ml |
| | (determined by HPLC, concentration of solubility). |
| Test solution #2: | Solution of the compound of formula (Ia) in a mixture of toluene |
| | and DMSO 80:20 (V/V). Solution was prepared the day before |
| | study by adding 1236.2 mg of the compound of formula (Ia) to 25 ml |
| | of a mixture of toluene and DMSO 80:20 (V/V) (49.5 mg/ml |
| | saturised solution). Concentration was 5.4 mg/ml (determined by |
| | HPLC, concentration of solubility). |
| Test solution #3: | Solution of the compound of formula (Ia) in a mixture of |
| | isopropylmyristate and Pharmasolve ™ 80:20 (V/V). Solution was |
| | prepared the day before study by adding 550.4 mg of the |
| | compound of formula (Ia) to 25 ml of a mixture of |
| | isopropylmyristate and Pharmasolve ™ 80:20 (V/V) (22 mg/ml |
| | saturised solution. Concentration was 0.052 mg/ml (determined |
| | by HPLC, concentration of solubility). |

Analytics

Samples were transferred into brown glass HPLC-vials and stored at −20° C. prior to analysis.

HPLC Parameters

| Column: | Nucleosil C-18, 250 × 3 mm, 5 μm |
|---|---|
| Column temperature: | 40° C. |
| Mobile phase A: | 0.01M Ammonium formiate (dissolve 0.63 g |
| | ammonium formiate in 1000 ml water) |
| Mobile phase B: | Acetonitrile |
| Gradient program: | t_{o′} 95% A/5% B |
| | t_{10′} 95% B, linear increase |
| | t_{15′} 95% B, isocratic |
| | t_{17′} 5% B, linear decrease |
| | t_{25′} Stop |
| Flow rate: | 0.5 ml/min |
| Injection volume: | 50 μl |
| Detection: | 290 nm |
| Retention time: | the compound of formula (Ia) approx. 5.9 min |
| Stop time: | 25 min |
| Number of injections: | 2 |

Quality Assurance

All experimental work was done according to the quality requirements outlined by the EG-cGMP rules. The used test equipment is monitored at regular periods and checked against national and international standards.

All chemicals and reagents used were at the time of analysis within the shelf-life of the time specified by the manufacturer. Raw material of the tested drug was material synthesized as described above in Example 1, set to 100% and used as directed.

Sink conditions were maintained throughout the whole experiment (Solubility in the acceptor medium was at least 10 times higher than the amount measured after 72 hours of the best permeating test-solution). Since solution #2 contained the highest drug loading with 49.5 mg/ml, the absolute permeated amount measured in the acceptor was 0.24 mg/ml, which was a factor of 200 lower, thus sink conditions were maintained. The saturation concentration of the compound of formula (Ia) in the acceptor medium was 130 mg/ml, which was a factor of 540 more than the amount that permeated. The limit of detection was determined to be 0.12 μg/ml. The tightness of the cells and absence of air bubbles in the receptor phase was monitored and given at each sampling point.

All drug containing solutions were stored protected from light. The solutions were stable regarding purity for 72 hours at 32° C. in comparison to start, only the content increased slightly; probably due to solubilisation of more API.

Calculation of Values from Raw Data

Calculation of Saturation Factor

In all cases, where different permeability rates were to be compared and different solubility in the applied vehicles were expected, all solutions had to have an infinite dose, i.e. there had to be a sufficient amount of the drug in the donor solution even if high amounts were penetrating. This was achieved by application of saturated solutions, which were solutions containing higher levels of the drug than was soluble (i.e. being a suspension). So, from manufacturing of the solutions, the amount of the compound of formula (Ia) per solvent volume was known (documentation by balance print out). That amount was named a saturated solution. Afterwards the saturated solution was centrifuged and the solubility concentration of the clear supernatant of the solution was determined quantitatively by HPLC. The saturation factor was calculated from the ratio of saturated solution:solubility concentration. A high ratio represented a high level of infinite dose.

Calculation of Flux Rate in [μg/Cm²/h] and Lag-Time

The flux was calculated for every formulation by means of linear regression (performance with Excel software; regression coefficient to be >0.98) from the mean cumulative permeated amounts in μg/cm² during steady state against the time in hours. For test solution #1 the linear regression was calculated from 12-48 hours (not until 72 hours, because after 48 hours the permeation was decreasing). For test solution #2 the linear regression was calculated from 24-72 hours (because of the long lag-time steady state started later than 12 hours). For test solution #3 the linear regression was calculated over the whole time from 12-72 hours. The lag-time (which was the time when steady state of permeation was starting after application) was taken from the intersection of the regression line with the time axis.

Calculation of Enhancement Factor

The enhancement factor was the ratio of flux rates in μg/cm²/h from test solution #1 (control vehicle): test solution #2 or #3 (vehicle with enhancers). The higher the factor, the more potent was the used enhancer composition.

Calculation of Assumed Transdermal Delivery Rates

The transdermal delivery rate was assumed to be the same in vivo as it is in vitro (this must, of course, be proven by further kinetic studies). The patch size was set to 20 cm², which is middle sized (small patches are 5 cm²; big patches are 60 cm²) and the delivery rate was calculated daily (1 day=24 hours).

In total:

(flux rate steady state [μg/cm²/h]×20 [cm²]×24[h])/1000=delivery rate [mg/20 cm²/24 h].

Delivery rate was expressed in [mg per patch per day].

Results

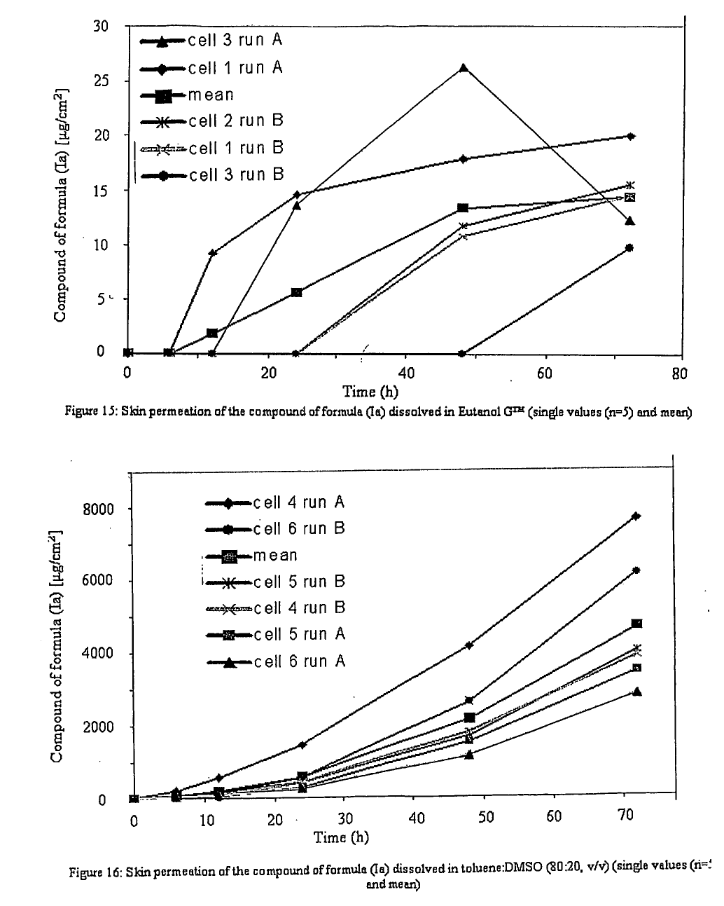

The results are summarized in FIGS. 15-18.

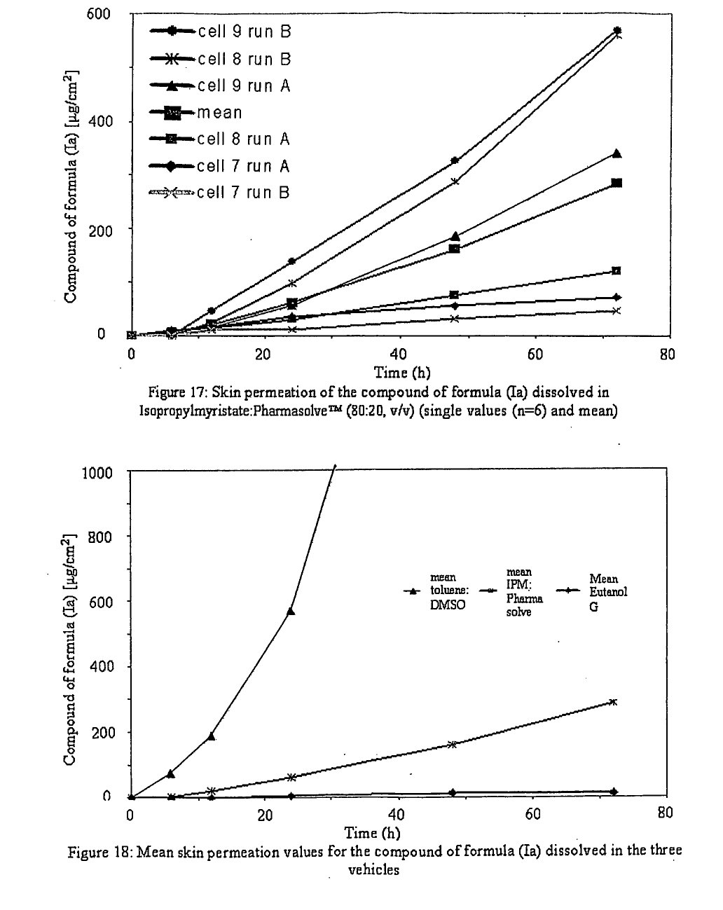

FIG. 15 shows the single values and the mean of test solution #1 (the compound of formula (Ia) dissolved in Eutanol G™). FIG. 16 shows the single values and the mean of test solution #2 (the compound of formula (Ia) dissolved in toluene: DMSO). FIG. 17 shows the single values and the mean of test solution #3 (the compound of formula (Ia) dissolved in Isopropylmyristate:Pharmasolve™). In FIG. 18 the mean values from the three solvents are shown together.

Test Solution #1

The lag time (time until steady state fluxes are reached) was 6 hours, which was quite usual for topical application. The steady state fluxes were calculated as 0.318 μg/cm²/h. If the flux in vitro were identical to that in vivo and the treated area was assumed to be 20 cm², 0.15 mg per day of the compound of formula (Ia) would be delivered to the body.

This solution was taken as “control”. Originally water was planned, however, due to the high water solubility of the compound, Eutanol G (2-Octyl-dodecanol) was used, which is not reported as an enhancer, but with good compatibility on the skin.

One of the six results was eliminated from the evaluation. This cell had a factor of 10 higher permeation, thus increasing the mean for the other 5 markedly. A small anomaly in the skin was recognized after the experiment, which is considered to be the reason for a falsely high result and thus this cell was eliminated as an outlier.

Test Solution #2

The lag time (time until steady state fluxes were reached) was 18 hours, which was quite long for topical application. The steady state fluxes were calculated as 85.09 μg/cm²/h. If the flux in vitro were identical to that in vivo and the treated area was assumed to be 20 cm², 40.8 mg per day of the compound of formula (Ia) would be delivered to the body. Originally the solution should have contained a water: DMSO mixture of 30:70. However, due to the high solubility of the compound of formula (Ia) in both media the amount of DMSO was reduced to 20%. A medium miscible with DMSO and with less solubility than water was desired. The only one we could rapidly identify without too much consumption of active was toluene. Vehicles like PEG 400, paraffin, n-hexane and the addition of tenside Tween 80 were unsuccessful. Toluene is not usually used as a vehicle on skin in larger amounts, since it depletes the skin of fatty acids. Fatty acids are necessary for barrier function of the stratum corneum. It must be assumed, that for a hydrophilic drug like the compound of formula (Ia) the stratum corneum will be the strongest barrier, which was impaired here by the toluene. It is therefore probable that this permeation result shows more or less the maximum enhancement possible for the compound of formula (Ia).

Test Solution #3:

The lag time (time until steady state fluxes were reached) was 9 hours, which was quite usual for a topical application. The steady state fluxes were calculated as 4.4 μg/cm²/h. If the flux in vitro were be identical to that in vivo and the treated area was assumed to be 20 cm², 2.1 mg per day of the compound of formula (Ia) would be delivered to the body.

Originally the vehicle was planned to be a tertiary mixture of Ethanol, Isopropylmyristate (IPM) and N-methyl-2-pyrrolidone (Pharmasolve) in the ratio of 27:64:9. This mixture was taken from the literature (Suwanipidokkul N. et al.). The mixture was tested as an enhancer for transdermal delivery of Zidovudine (AZT) in pig skin and it was found to be effective for therapeutic purposes. AZT is a 3′-azido-3′-deoxythymidine, a hydrophilic molecule of comparable solubility and molecular weight to the compound of formula (Ia) and therefore these enhancers were considered to be worth testing. However, due to the high solubility of the compound of formula (Ia) in Ethanol, the mixture was changed to a binary mixture, i.e. IPM:Pharmasolve 80:20. Both excipients can be incorporated into base formulations of a cream as well as of a self-adhesive transdermal patch.

### REFERENCES

Mills I. W. (1999) The pathophysiology of detrusor instability and the role of bladder ischaemia in its aetiology. University of Oxford DM thesis: 32
Suwanipidokkul N. et al, (2004) AAPS Pharm. Sci. Tech. 5(3)

1. A compound of formula (I) or a pharmaceutically acceptable solvate or salt thereof, including a solvate of such a salt, 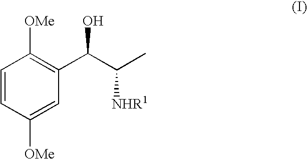
wherein R¹ is a group such that the compound of formula (I) is a prodrug of 1R,2S-methoxamine that is converted within the kidney tubules into its active form or an active metabolite thereof.

2. A compound as claimed in claim 1 wherein R¹ is a group that is capable of being cleaved by an amino transferase enzyme.

3. A compound as claimed in claim 2 wherein R¹ is a group that is capable of being cleaved by gamma glutamyl transferase.

4. A compound as claimed in any of claims 1 to 3 wherein the R¹ group is linked by an amide bond to the amino group of 1R,2S-methoxamine.

5. A compound as claimed in claim 1 wherein the compound of formula (I) is the compound of formula (Ia) or a pharmaceutically acceptable solvate or salt thereof, including a solvate of such a salt

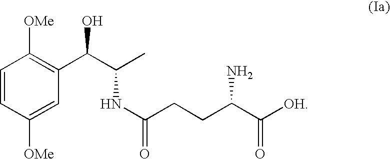

6. (canceled)

7. (canceled)

8. (canceled)

9. A pharmaceutical composition comprising a compound as claimed in claim 1, and a pharmaceutically acceptable excipient or carrier.

10. A pharmaceutical composition as claimed in claim 9 that is in a form suitable for administration by a route other than oral.

11. A pharmaceutical composition as claimed in claim 9 in a form suitable for transdermal administration.

12. A pharmaceutical composition as claimed in claim 11 comprising a skin permeation enhancer.

13. A pharmaceutical composition as claimed in claim 12 where the skin permeation enhancer is a mixture of isopropylmyristate and Pharmasolve™ (N-methyl pyrrolidone).

14. A method of treating urinary incontinence, which comprises administering a therapeutically effective amount of a compound as claimed in claim 1 or claim 5 to a mammal in need of said treatment.

15. A method as claimed in claim 14, wherein the composition or compound is administered transdermally.

16. A process for the synthesis of a compound as claimed in claim 1 or claim 5 which comprises reacting 1R,2S-methoxamine with a compound of the formula R¹—OH, in the presence of a suitable coupling agent and optionally in the presence of a base, wherein R¹ is as defined in claim 1.

17. A process for the synthesis of a compound as claimed in claim 1 or claim 5 which comprises reacting 1R,2S-methoxamine with a compound of the formula R¹-L, optionally in the presence of a base, wherein R¹ is as defined in claim 1 and L is a suitable leaving group.

18. A process for the synthesis of a compound as claimed in claim 5, which comprises reacting a compound of formula (II) 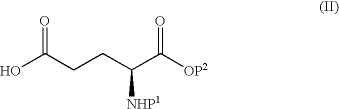
wherein P¹ and P² are suitable protecting groups with 1R,2S-methoxamine, in the presence of a suitable base and optionally in the presence of one or more suitable coupling agents, followed by removal of the protecting groups.

19. A method of treating urinary incontinence, which comprises administering a therapeutically effective amount of a pharmaceutical composition as claimed in claim 9.
